(19) United States
(12) Patent Application Publication
(10) Pub. No.: US 2005/0255069 A1
(43) Pub. Date: Nov. 17, 2005

(54) Cosmetic compositions comprising at least one salt, at least one cyclodextrin, and at least one surfactant, and uses thereof

(76) Inventor: Rainer Muller, Leopoldshafen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/116,215

(22) Filed: Apr. 28, 2005

Related U.S. Application Data
(60) Provisional application No. 60/567,783, filed on May 5, 2004.

(30) Foreign Application Priority Data
Apr. 28, 2004 (FR) 04 50813

(51) Int. Cl. A61K 7/06; A61K 7/11

(52) U.S. Cl. 424/70.13

(57) ABSTRACT

The present disclosure relates to compositions comprising, in a physiologically acceptable aqueous medium, at least one cyclodextrin, at least one surfactant, and at least one ammonium salt or a salt of a monovalent or divalent metal cation. The disclosure also relates to compositions further comprising at least one conditioning agent. The compositions according to the present disclosure may be used, for example, as rinse-out products, for instance for washing and/or conditioning keratin materials.

This application claims benefit of U.S. Provisional Application No. 60/567,783, filed May 5, 2004, the contents of which are incorporated herein by reference. This application also claims benefit of priority under 35 U.S.C. § 119 to French Patent Application No. 04 50813, filed Apr. 28, 2004, the contents of which are also incorporated by reference.

The present disclosure relates to compositions comprising, in a physiologically acceptable aqueous medium, at least one compound chosen from cyclodextrins and derivatives thereof, at least one surfactant, and at least one salt of a monovalent or divalent monomeric metal cation. The present disclosure also relates to such compositions where the composition further comprises at least one conditioning agent. The present disclosure further relates to the use of the at least one salt of a monovalent or divalent monomeric metal cation as an agent for suspending the complex formed from the at least one compound chosen from cyclodextrins and derivatives thereof and from the at least one surfactant.

It is well known that hair that has been sensitized (i.e. damaged and/or embrittled) to varying degrees due to the action of atmospheric agents or mechanical or chemical treatments, such as dyeing, bleaching and/or permanent-waving, may often be difficult to disentangle and to style, and may lack softness.

The use of conditioning agents, for example insoluble conditioning agents, that can facilitate disentangling of keratin fibers and make them soft, shiny and supple, has already been recommended in compositions for washing and/or caring for keratin materials such as the hair.

Given the insoluble nature of certain conditioning agents, for example silicones and oils, it would be desirable to find a way to maintain the conditioning agents in uniform dispersion in the medium without, however, causing a reduction in the viscosity and in the detergent and foaming properties of the compositions. Conditioning agents, for example silicones and oils, must also be able to be conveyed onto the treated keratin materials in order to give them, depending on the application, softness, sheen, and/or disentangling properties.

It is also known that products, such as cosmetic products, having an iridescent, moiré or metallic appearance or effect, are widely appreciated by consumers for their attractive appearance, which may give the product an appearance of richness. The agents that can provide this effect include nacreous agents or nacres, which may comprise crystals that can remain dispersed in the compositions and can reflect light.

Long-chain ester derivatives are widely used to make compositions, for example cosmetic compositions, nacreous. However, these derivatives may have crystallization problems that may cause a change in the viscosity of compositions over time.

Long-chain ether and thioether derivatives such as those described in European Patent Application No. EP 457 688 and International Patent Publication No. WO 98/03155 are also known. However, these agents can opacify compositions without, or without sufficiently, giving them a nacreous effect.

It has also been found that, because of their low density, these nacreous agents may have the drawback of rising to the surface of the shampoo and may form, as a result, a layer on the surface that consumers find unattractive.

Furthermore, in certain cases, these fatty-chain compounds may have the drawback of giving a laden feel to the hair, and a lack of lightness and volume to the hairstyle.

Moreover, these nacreous agents may be water-insoluble compounds and may have a melting point above 50° C. To manufacture nacreous compositions, the compositions may thus need to be heated above the melting point of the nacreous compound and then cooled, and the other compounds of the compositions may subsequently be added. In order to reduce energy consumption and manufacturing time, it is desirable to prepare nacreous compositions without heating.

International Patent Publication No. WO 03/088934 describes the use of cyclodextrin as a nacreous agent that can be used without heating. However, the compositions comprising a cyclodextrin may still not be sufficiently stable.

Stabilizers such as crosslinked acrylic polymers of the Carbopol type may be used to thicken and stabilize cosmetic compositions comprising insoluble conditioning agents. However, these stabilizers may have the drawback of reducing the cosmetic performance qualities of shampoos, for example by making the hair more laden and coarser.

The present inventor has discovered that it is possible to formulate compositions, for example cosmetic compositions for treating keratin materials, such as shampoos with a nacreous appearance, which have at least one of the above-described desired aesthetic and cosmetic properties, by using in these cosmetic compositions at least one compound chosen from cyclodextrins and derivatives thereof, at least one surfactant, and at least one salt.

One aspect of the present disclosure relates to compositions, for example cosmetic compositions, comprising, in a physiologically acceptable, for instance a cosmetically acceptable, aqueous medium, at least one anionic surfactant in an amount ranging from 1% to 35% by weight, relative to the total weight of the composition, at least one compound chosen from cyclodextrins and derivatives thereof in an amount ranging from 1% to 15% by weight, relative to the total weight of the composition, and at least one salt chosen from ammonium (NH₄⁺) salts and salts of monovalent or divalent metals and of a mineral acid or of an organic acid comprising from 1 to 7 carbon atoms in an amount ranging from 0.1% to 10% by weight, relative to the total weight of the composition.

The compositions may show very good homogeneity and good stability of the nacre, and also a viscosity that may be satisfactory for application to keratin materials. The compositions also may have no phase separation.

For example, the compositions disclosed herein may retain their texture over time, i.e., no uncontrolled graining out or thickening of the composition over time takes place. Finally, the compositions may have a non-runny, fondant texture. The lather may be easy to rinse out.

Another aspect of the present disclosure relates to washing and/or conditioning processes using compositions as disclosed herein.

Yet another aspect of the present disclosure relates to the use of a monomeric ammonium (NH₄⁺) salt or a salt of a monovalent or divalent metal cation in, or for the manufacture of, cosmetic compositions comprising at least one cyclodextrin and at least one surfactant.

Other aspects of the present disclosure will become apparent upon reading the description and the examples that follow.

Within the context of the present disclosure, the term “keratin materials” is understood to mean the hair, the eyelashes, the eyebrows, the skin, the nails, mucous membranes, and the scalp, for example the hair.

The terms “nacreous agent” and “nacre,” as used herein, are understood to mean an agent that produces a nacreous, iridescent, moiré or metallic appearance or effect.

Within the context of the present disclosure, the term “monomeric salt” is understood to mean that neither the cation nor the anion are oligomers or polymers.

For example, the at least one salt according to the present disclosure may be chosen from salts of monovalent or divalent metals and of a mineral acid or of an organic acid comprising from 1 to 7 carbon atoms.

The salts may be chosen, for example, from alkali metal salts, alkaline-earth metal salts, and transition metal salts. By way of non-limiting example, the salts that may be mentioned include lithium, sodium, potassium, magnesium, calcium, strontium and/or barium salts, and manganese, cobalt, and zinc salts, and mixtures thereof. In at least one embodiment, the salts may be chosen, for example, from sodium, potassium, magnesium, calcium, manganese, and zinc salts, and as a further example, from sodium salts.

The mineral or organic acids may be chosen from, for example, carbonates, bicarbonates, sulfates, glycerophosphates, borates, chlorides, nitrates, acetates. Also useful are salts of hydroxy acids, such as gluconate, salts of fruit acids, such as citrate, tartrate, lactate, and malate, and salts of amino acids, such as aspartate, arginate, glucocholate, and fumarate.

For example, the at least one salt may be chosen from chlorides, sulfates, gluconates, acetates, phosphates, and citrates.

In at least one embodiment, the at least one salt is chosen from sodium chloride, potassium chloride, calcium chloride, magnesium sulfate, magnesium gluconate, and sodium citrate. For example, the at least one salt may be sodium chloride.

The at least one salt may be present in the disclosed compositions in an amount ranging from 0.1% to 10% by weight, for example from 0.5% to 5% by weight, relative to the total weight of the composition.

The at least one compound chosen from cyclodextrins and derivatives thereof may be an oligosaccharide of formula: 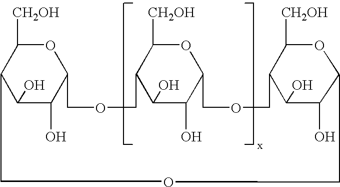
wherein x may be a number equal to 4 (which corresponds to α-cyclodextrin), to 5 (β-cyclodextrin) or to 6 (γ-cyclodextrin).

Non-limiting examples of cyclodextrins that may be used in compositions according to the present disclosure include a β-cyclodextrin sold by the company Wacker under the name CAVAMAX W7 and a γ-cyclodextrin sold by the company Wacker under the name CAVAMAX W8.

The cyclodextrin derivatives may be, for example, methylcyclodextrins such as the methyl-β-cyclodextrin sold by the company Wacker under the name CAVASOL W7.

According to the present disclosure, the at least one compound chosen from cyclodextrins and derivatives thereof may be present in an amount from 1% to 15% by weight, for example from 1% to 10% by weight, such as from 1.5% to 5% by weight, relative to the total weight of the final composition.

The compositions according to the present disclosure may comprise at least one anionic surfactant, which may be present in an amount ranging from 1% to 35% by weight, for example from 4% to 35% by weight, such as from 8% to 30% by weight, relative to the total weight of the composition.

The at least one compound chosen from cyclodextrins and derivatives thereof and the at least one surfactant may be present at a concentration that is effective in making the composition nacreous and/or in forming an insoluble complex in the composition between the at least one compound and the at least one surfactant.

For example, the at least one compound chosen from cyclodextrins and derivatives thereof may be introduced into the composition in non-complexed form or optionally complexed with the at least one surfactant, such that when a complex is made, it is not made with a compound other than the at least one surfactant.

The at least one compound chosen from cyclodextrins and derivatives thereof may be complexed into the composition with the at least one surfactant.

The ratio of the at least one surfactant to the at least one compound chosen from cyclodextrins and derivatives thereof may range from 0.01 to 300, for example from 0.1 to 100, such as from 0.3 to 25.

The surfactants suitable for use in the compositions according to the present disclosure may be of any nature, and may be, for example, soluble in water at room temperature:

(i) Anionic Surfactants:

In the context of the present disclosure, the nature of the at least one anionic surfactant is not a truly critical feature.

Thus, among the anionic surfactants that can be used, alone or as mixtures, in the compositions according to the present disclosure, non-limiting mention may be made, for example, of salts, such as alkaline salts, for instance sodium salts, ammonium salts, amine salts, amino alcohol salts, and magnesium salts, of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates, and N-acyltaurates, wherein the alkyl or acyl radical of these various compounds may comprise from 8 to 24 carbon atoms, and wherein the aryl radical may be chosen from phenyl and benzyl groups. Among the anionic surfactants that can be used in the compositions according to the present disclosure, further non-limiting mention may be made of fatty acid salts such as the salts of oleic, ricinoleic, palmitic, and stearic acids, coconut oil acid and hydrogenated coconut oil acid; and acyl lactylates in which the acyl radical may comprise from 8 to 20 carbon atoms. The anionic surfactants may also be chosen from weakly anionic surfactants, such as alkyl-D-galactosiduronic acids and their salts, as well as polyoxyalkylenated (C₈-C₂₄) alkyl ether carboxylic acids, polyoxyalkylenated (C₈-C₂₄) alkylaryl ether carboxylic acids, polyoxyalkylenated (C₈-C₂₄) alkylamido ether carboxylic acids and their salts, for example those comprising from 2 to 50 ethylene oxide groups, and mixtures thereof.

For example, the at least one anionic surfactant that may be used in compositions according to the present disclosure include alkyl sulfate salts, alkyl ether sulfate salts, and mixtures thereof.

In addition to the at least one anionic surfactant, the compositions may further comprise surfactants chosen from nonionic, amphoteric, and cationic surfactants. For example, the additional surfactants may be amphoteric and/or nonionic.

(ii) Nonionic Surfactant(s):

The nonionic surfactants are compounds that are well known per se (in this respect, see, for example, “Handbook of Surfactants” by M. R. Porter, published by Blackie & Son (Glasgow and London), 1991, pp. 116-178) and, in the context of the present disclosure, their nature is not a critical feature. Thus, non-limiting examples of non-ionic surfactants that may be used in compositions according to the present disclosure include polyethoxylated, polypropoxylated, and polyglycerolated fatty acids, alkylphenols, α-diols, and alcohols, wherein these compounds comprise a fatty chain comprising, for example, from 8 to 18 carbon atoms, wherein the number of ethylene oxide or propylene oxide groups may range, for example, from 2 to 50, and wherein the number of glycerol groups may range, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising 1 to 5, for example 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides, and N-acylaminopropylmorpholine oxides. For example, the non-ionic surfactants may be chosen from alkylpolyglycosides.

(iii) Amphoteric or Zwitterionic Surfactant(s):

Non-limiting examples of amphoteric and zwitterionic surfactants, the nature of which is not a critical feature in the context of the present disclosure, include aliphatic secondary and tertiary amine derivatives in which the aliphatic radical may be chosen from linear and branched chains comprising from 8 to 18 carbon atoms and comprising at least one water-soluble anionic group, such as carboxylate, sulfonate, sulfate, phosphate, and phosphonate; non-limiting mention may also be made of (C₈-C₂₀)alkylbetaines, sulfobetaines, (C₈-C₂₀)alkylamido(C₁-C₆)alkylbetaines, and (C₈-C₂₀)alkylamido(C₁-C₆)alkylsulfobetaines.

Among the amine derivatives, non-limiting mention may be made of the products sold under the name MIRANOL, as described in U.S. Pat. Nos. 2,528,378 and 2,781,354, and classified in the CTFA dictionary, 3^{rd} edition, 1982, under the names Amphocarboxy glycinates and Amphocarboxy propionates, and having the respective structures:
R₂—CONHCH₂CH₂—N(R₃)(R₄)(CH₂COO—) (2)
wherein:
R₂ is chosen from alkyl radicals derived from an acid R₂—COOH present in hydrolyzed coconut oil; and heptyl, nonyl, and undecyl radicals;
R₃ is chosen from β-hydroxyethyl groups; and
R₄ is chosen from carboxymethyl groups;
   and
   R_{2′}—CONHCH₂CH₂—N(B)(C) (3)
   wherein:
B is chosen from groups of formula —CH₂CH₂OX′, wherein X′ is chosen from a —CH₂CH₂—COOH group and a hydrogen atom,
C is chosen from radicals of formula —(CH₂)ₙ—Y′, wherein:
   z equals 1 or 2, and
   Y′ is chosen from —COOH and —CH₂—CHOH—SO₃H radicals, and
R₂ is chosen from alkyl radicals derived from groups of formula R₂, —COOH present in coconut oil and in hydrolyzed linseed oil, alkyl radicals, such as C₇, C₉, C₁₁, and C₁₋₃ alkyl radicals, C₁₋₇ alkyl radicals and their iso form, an unsaturated C₁₇ radical.

For example, non-limiting mention may be made of the cocoamphodiacetate sold under the trade name MIRANOL C2M concentrated NP by the company Rhodia Chimie.

(iv) Cationic Surfactants:

The cationic surfactants may be chosen from:

A) the quaternary ammonium salts of formula (XII): 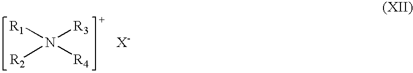
wherein:
X⁻ is an anion that may be chosen from halides, such as chloride, bromide, and iodide; (C₂-C₆)alkyl sulfates, such as methyl sulfate; phosphates; alkyl and alkylaryl sulfonates; and anions derived from organic acids, such as acetate and lactate;
   i) the radicals R₁, R₂, and R₃, which may be identical or different, are chosen from linear and branched aliphatic radicals comprising from 1 to 4 carbon atoms, and aromatic radicals such as aryl and alkylaryl radicals. The aliphatic radicals can comprise at least one heteroatom chosen from, for example, oxygen, nitrogen, sulfur, and halogens. The aliphatic radicals may be chosen, for example, from alkyl, alkoxy, and alkylamide radicals,
R₄ may be chosen from linear and branched alkyl radicals comprising from 16 to 30 carbon atoms.

For example, the cationic surfactant may be chosen from salts, for example chloride, of cetyltrimethylammonium salts.
ii) the radicals R₁ and R₂, which may be identical or different, may be chosen from linear and branched aliphatic radicals comprising from 1 to 4 carbon atoms and aromatic radicals such as aryl and alkylaryl. The aliphatic radicals can comprise at least one heteroatom chosen, for example, from oxygen, nitrogen, sulfur, and halogens. The aliphatic radicals may be chosen, for example, from alkyl, alkoxy, alkylamide, and hydroxyalkyl radicals comprising from 1 to 4 carbon atoms;
R₃ and R₄, which may be identical or different, may be chosen from linear and branched alkyl radicals comprising from 12 to 30 carbon atoms, wherein the radicals may comprise at least one group chosen from ester functional groups and amide functional groups.

R₃ and R₄ may be chosen, for example, from (C₁₂-C₂₂)alkylamido(C₂-C₆)alkyl and (C₁₂-C₂₂)alkylacetate radicals.

For example, the cationic surfactant may be a stearamidopropyldimethyl(myristyl acetate)ammonium salt, such as a chloride of stearamidopropyldimethyl(myristyl acetate)ammonium.

B) quaternary ammonium salts of imidazolinium, such as, for example, that of formula (XIII): 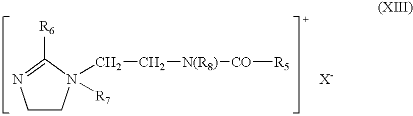
wherein:
R₅ is chosen from alkenyl and alkyl radicals comprising from 8 to 30 carbon atoms, such as fatty acid derivatives of tallow;
R₆ is chosen from a hydrogen atom, C₁-C₄ alkyl radicals, and alkenyl and alkyl radicals comprising from 8 to 30 carbon atoms;
R₇ is chosen from C₁-C₄ alkyl radicals;
R₈ is chosen from a hydrogen atom and C₁-C₄ alkyl radicals; and
X⁻ is an anion chosen from halides, phosphates, acetates, lactates, alkyl sulfates, alkyl sulfonates, and alkylaryl sulfonates.

For example, R₅ and R₆ may be chosen from mixtures of alkenyl and alkyl radicals comprising from 12 to 21 carbon atoms, such as, for example, fatty acid derivatives of tallow, R₇ may be a methyl radical, and R₈ may be a hydrogen atom. Such products include, for example, Quaternium-27 (CTFA 1997) and Quaternium-83 (CTFA 1997), which are sold under the names “REWOQUAT” W75, W90, W75PG, and W75HPG by the company Witco.

C) the diquaternary ammonium salts of formula (XIV): 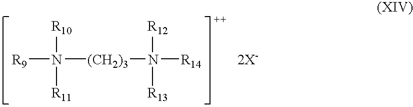
wherein:
R₉ is chosen from aliphatic radicals comprising from 16 to 30 carbon atoms;
R₁₀, R₁₁, R₁₂, R₁₃, and R₁₄, which may be identical or different, are chosen from a hydrogen atom and alkyl radicals comprising from 1 to 4 carbon atoms; and
X⁻ is an anion chosen from halides, acetates, phosphates, nitrates, and methyl sulfates. Such diquaternary ammonium salts may, for example, be propane tallow diammonium dichloride.

D) the quaternary ammonium salts comprising at least one ester functional group, of formula (XV): 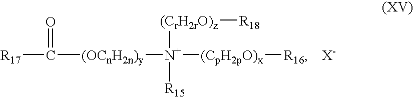
wherein:
R₁₅ is chosen from C₁-C₆ alkyl radicals and C₁-C₆ hydroxyalkyl and dihydroxyalkyl radicals;
R₁₆ is chosen from:
   the radical 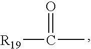
   the radical R₂₀, wherein R₂₀ is chosen from linear and branched, saturated and unsaturated C₁-C₂₂ hydrocarbon radicals, and
   a hydrogen atom,
R₁₈ is chosen from:
   the radical 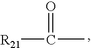
   the radical R₂₂, wherein R₂₂ is chosen from linear and branched, saturated and unsaturated C₁-C₆ hydrocarbon radicals, and
   a hydrogen atom,
R₁₇, R₁₉ and R₂₁, which may be identical or different, are chosen from linear and branched, saturated and unsaturated C₇-C₂₁ hydrocarbon radicals;
n, p and r, which may be identical or different, are chosen from integers ranging from 2 to 6;
y is chosen from integers ranging from 1 to 10;
x and z, which may be identical or different, are chosen from integers ranging from 0 to 10; and
X⁻ is an anion chosen from simple and complex, organic and inorganic anions; with the proviso that the sum x+y+z is equal to an integer ranging from 1 to 15, that when x is equal to 0, then R₁₆ is R₂₀, and that when z is equal to 0, then R₁₈ is R₂₂.

For example, ammonium salts of formula (XV) that may be used in compositions according to the present disclosure include those in which:
R₁₅ is chosen from methyl and ethyl radicals,
x and y are equal to 1;
z is equal to 0 or 1;
n, p, and r are equal to 2;
R₁₆ is chosen from:
   the radical 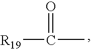
   methyl, ethyl, and C₁₄-C₂₂ hydrocarbon radicals, and
   a hydrogen atom;
R₁₇, R₁₉, and R₂₁, which may be identical or different, are chosen from linear and branched, saturated and unsaturated C₇-C₂, hydrocarbon radicals; and
R₁₈ is chosen from:
   the radical 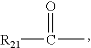
   and
   a hydrogen atom.

Such compounds are sold, for example, under the names DEHYQUART by the company Cognis, STEPANQUAT by the company Stepan, Noxamium by the company Ceca, and REWOQUAT WE 18 by the company Degussa-Witco.

Among the quaternary ammonium salts that may be used in compositions according to the present disclosure, non-limiting mention may be made of cetyltrimethylammonium chloride and palmitamidopropyltrimethylammonium chloride, sold under the name VARISOFT PA TC by the company Degussa.

The compositions may comprise at least one anionic surfactant.

For example, the anionic surfactants that may be used in compositions according to the present disclosure include sodium, triethanolamine and ammonium (C₁₂-C₁₄)alkyl sulfates, sodium, triethanolamine and ammonium (C₁₂-C₁₄)alkyl ether sulfates oxyethylenated with 2.2 mol of ethylene oxide, sodium cocoyl isethionate and sodium α-(C₁₄-C₁₆)olefin sulfonate, and their mixtures with:
either at least one amphoteric surfactant, chosen, for example, from the amine derivatives known as disodium cocoamphodiacetate and sodium cocoamphopropionate, sold, for example, by the company Rhodia Chimie under the trade name Miranol® C2M CONCNP as an aqueous solution comprising 38% active material, and under the name Miranol® C32;
or at least one amphoteric surfactant, chosen, for example, from alkylbetaines, such as the cocobetaine sold under the name Dehyton® AB 30 as an aqueous solution comprising 32% AM by the company Cognis, and (C₈-C₂₀)alkylamido(C₁-C₆)alkylbetaines, such as Tegobetaine® F 50 sold by the company Degussa.

According to one embodiment, the cosmetic compositions according to the present disclosure may further comprise agents for conditioning keratin materials.

When these compositions are applied to the hair, they may have good hair conditioning properties, i.e., the treated hair may be smooth, disentangle easily, and/or feel soft. The hair may have a natural, unladen appearance.

When the compositions comprise at least one conditioning agent, the at least one conditioning agent may be chosen from synthetic oils such as polyolefins, for example poly-α-olefins, fluoro oils, fluoro waxes, fluoro gums, carboxylic acid esters, cationic polymers, silicones, mineral, plant and animal oils, ceramides, pseudoceramides, and mixtures thereof.

The polyolefins may be chosen, for example, from poly-α-olefins, such as: hydrogenated and non-hydrogenated polybutene poly-α-olefins, for instance hydrogenated and non-hydrogenated polyisobutene poly-α-olefins. For example, isobutylene oligomers with a molecular weight of less than 1000 and mixtures thereof with polyisobutylenes with a molecular weight of greater than 1000, such as polyisobutylenes with a molecular weight ranging from 1000 to 15 000, may be used.

As examples of poly-α-olefins that may be used in compositions according to the present disclosure, non-limiting mention may be made of the polyisobutenes sold under the name PERMETHYL 99 A, 101 A, 102 A, 104 A (n=16) and 106 A (n=38) by the company Presperse Inc., and the products sold under the name ARLAMOL HD (n=3) by the company ICI (n denoting the degree of polymerization).
and hydrogenated and non-hydrogenated polydecene poly-α-olefins.

Such products are sold, for example, under the names ETHYLFLO by the company Ethyl Corp. and ARLAMOL PAO by the company ICI.

The mineral oils that may be used in the compositions according to the present disclosure may be chosen, for example, from hydrocarbons, such as hexadecane and liquid paraffin.

The cationic polymers that may be used in the compositions according to the present disclosure may be chosen from those already known per se as improving the cosmetic properties of keratin fibers treated with detergent compositions, for example those described in European Patent Application No. EP-A-0 337 354 and in French Patent Application Nos. FR-A-2 270 846, 2 383 660, 2 598 611, 2 470 596, and 2 519 863.

Within the context of the present disclosure, the term “cationic polymer” is understood to mean any polymer comprising cationic groups and/or groups that may be ionized into cationic groups.

Among the cationic polymers that may be used in the compositions according to the present disclosure, non-limiting mention may be made of quaternary cellulose ether derivatives, such as the products sold under the name JR 400 by the company Amerchol, cyclopolymers, such as the diallyidimethylammonium salt homopolymers and the copolymers of a diallyldimethylammonium salt and of acrylamide, for instance, the chlorides, sold under the names MERQUAT 100, MERQUAT 550, and MERQUAT S by the company Nalco, cationic polysaccharides, such as guar gums modified with 2,3-epoxypropyltrimethylammonium chloride, sold, for example, under the name JAGUAR C13S by the company Meyhall, optionally crosslinked homopolymers and copolymers of a (meth)acryloyloxyethyltrimethylammonium salt, sold by the company Ciba as a 50% solution in mineral oil, under the trade names SALCARE SC92 (crosslinked copolymer of methacryloyloxyethyltrimethylammonium chloride and of acrylamide) and SALCARE SC95 (crosslinked homopolymer of methacryloyloxyethyltrimethylammonium chloride).

It may also be possible to use polymers comprising repeating units corresponding to the formula: 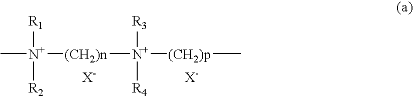
wherein:
R₁, R₂, R₃, and R₄, which may be identical or different, are chosen from alkyl and hydroxyalkyl radicals comprising from 1 to 4 carbon atoms;
n and p, which may be identical or different, are chosen from integers ranging from 2 to 20; and
X⁻ is an anion chosen from anions derived from mineral and organic acids.

The silicones that may be used in compositions according to the present disclosure include, for example, polyorganosiloxanes that are insoluble in the compositions and that may be in the form of oils, waxes, resins, and gums.

The water-insoluble silicones may be insoluble in water at a concentration of greater than or equal to 0.1% by weight in water at 25° C., i.e., they do not form a transparent isotropic solution.

The viscosity of the silicones may be measured, for example, at 25° C. according to ASTM standard 445 Appendix C.

For example, the polyorganosiloxanes are defined in greater detail in Walter Noll's “Chemistry and Technology of Silicones” (1968) Academic Press. They may be chosen from volatile and non-volatile polyorganosiloxanes.

Volatile silicones may be chosen, for example, from those having a boiling point ranging from 60° C. to 260° C., such as from:
(i) cyclic silicones comprising from 3 to 7, for example from 4 to 5, silicon atoms. Examples of these silicones include octamethylcyclotetrasiloxane sold, for instance, under the names VOLATILE SILICONE 7207 by Union Carbide and SILBIONE 70045 V 2 by Rhodia Chimie, decamethylcyclopentasiloxane sold under the names VOLATILE SILICONE 7158 by Union Carbide and SILBIONE 70045 V 5 by Rhodia Chimie, and mixtures thereof.

Further non-limiting mention of cyclic silicones may be made of cyclocopolymers of the dimethylsiloxane/methylalkylsiloxane type, such as SILICONE VOLATILE FZ 3109 sold by the company Union Carbide, having the chemical structure:

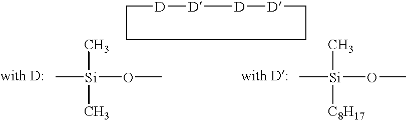

Non-limiting examples of silicones that may be used in compositions according to the present disclosure also include mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane.
(ii) linear volatile silicones comprising from 2 to 9 silicon atoms and having a viscosity of less than or equal to 5×10⁻⁶ m²/s at 25° C. For example, decamethyltetrasiloxane sold under the name SH 200 by the company Toray Silicone may be used. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, for example polyalkylsiloxanes, polyarylsiloxanes, polyalkylarylsiloxanes, silicone gums and resins, polyorganosiloxanes modified with organofunctional groups, and mixtures thereof, may be used in compositions according to the present disclosure.

These silicones may be chosen, for example, from polyalkylsiloxanes, among which non-limiting mention may be made of polydimethylsiloxanes comprising trimethylsilyl end groups having a viscosity of from 5×10⁻⁶ to 2.5 m²/s at 25° C., such as from 1×10⁻⁵ to 1 m²/s.

Among these polyalkylsiloxanes, non-limiting mention may be made of the following commercial products:
the oils of the Mirasil series sold by the company Rhodia Chimie, such as the oil MIRASIL DM 500 000;
the oils of the 200 series from the company Dow Corning, such as DC200 with a viscosity of 60 000 cSt;
the Viscasil oils from General Electric and certain oils of the SF series, such as SF 96 and SF 18 from General Electric.

Non-limiting mention may also be made of polydimethylsiloxanes comprising dimethylsilanol end groups (Dimethiconol according to the CTFA name), such as the oils of the 48 series from the company Rhodia Chimie.

In this category of polyalkylsiloxanes, non-limiting mention may also be made of the products sold under the names ABIL WAX 9800 and 9801 by the company Degussa, which are poly(C₁-C₂₀)alkylsiloxanes.

The polyalkylarylsiloxanes may be chosen, for example, from linear and branched polydimethylmethylphenylsiloxanes and polydimethyldiphenylsiloxanes, with a viscosity ranging from 1×10⁻⁵ to 5×10⁻² m²/s at 25° C.

Among these polyalkylarylsiloxanes, non-limiting mention may be made of the products sold under the following names:
MIRASIL DPDM oils from Rhodia Chimie;
oils of the RHODORSIL 70 633 and 763 series from Rhodia Chimie;
Dow Corning 556 Cosmetic Grade Fluid from Dow Corning;
silicones of the PK series from Bayer, such as the product PK20;
silicones of the PN and PH series from Bayer, such as the products PN1000 and PH1000; and
certain oils of the SF series from General Electric, such as SF 1023, SF 1154, SF 1250, SF 1265.

The silicone gums that can be used in compositions according to the present disclosure include, for example, polydiorganosiloxanes having high number-average molecular masses ranging from 200 000 to 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecane, and mixtures thereof.

Non-limiting mention may be made, for example, of the following products:
polydimethylsiloxane,
polydimethylsiloxane/methylvinylsiloxane gums,
polydimethylsiloxane/diphenylsiloxane,
polydimethylsiloxane/phenylmethylsiloxane, and
polydimethylsiloxane/diphenylsiloxane/methylvinylsiloxane.

Further non-limiting examples of silicones that may be used in compositions according to the present disclosure include mixtures such as:
mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 SILICONE FLUID from the company General Electric; this product is an SF 30 gum corresponding to a dimethicone, having a number-average molecular weight of 500 000, dissolved in the oil SF 1202 SILICONE FLUID corresponding to decamethylcyclopentasiloxane; and
mixtures of two PDMSs of different viscosities, for example, of a PDMS gum and a PDMS oil, such as the product SF 1236 from the company General Electric. The product SF 1236 is a mixture of an SF 30 gum defined above, having a viscosity of 20 m²/s, and an SF 96 oil, with a viscosity of 5×10⁻⁶ m²/s. This product, for example, comprises 15% SF 30 gum and 85% SF 96 oil.

The organopolysiloxane resins that can be used in compositions according to the present disclosure may be crosslinked siloxane systems comprising the units: (R)₂SiO_{2/2}, (R)₃SiO_{1/2}, RSiO_{3/2} and SiO_{4/2}, wherein R is chosen from hydrocarbon groups comprising from 1 to 16 carbon atoms and phenyl groups. Among these products, non-limiting mention may be made of the ones in which R is chosen from C₁-C₄ lower alkyl radicals, for example methyl, and phenyl radicals.

Among these resins, non-limiting mention may be made of the product sold under the name Dow Corning 593 and those sold under the names SILICONE FLUID SS 4230 and SS 4267 by the company General Electric, which are silicones of dimethyl/trimethyl siloxane structure.

Further non-limiting mention may be made of the trimethyl siloxysilicate type resins sold, for example, under the names X22-4914, X21-5034, and X21-5037 by the company Shin-Etsu.

The organomodified silicones that can be used in compositions according to the present disclosure include silicones as defined above and comprising in their structure at least one organofunctional group attached via a hydrocarbon radical.

Among the organomodified silicones, non-limiting mention may be made of polyorganosiloxanes comprising at least one organofunctional group chosen from:
polyethyleneoxy and polypropyleneoxy groups optionally comprising at least one C₆-C₂₄ alkyl group, such as the products known as dimethicone copolyol sold by the company Dow Corning under the name DC 1248 and the oils SILWET L 722, L 7500, L 77, and L 711 sold by the company Union Carbide, and the (C₁₂)alkylmethicone copolyol sold by the company Dow Corning under the name Q2 5200;
substituted and unsubstituted amine groups, such as the products sold under the name GP 4 SILICONE FLUID and GP 7100 by the company Genesee, and the products sold under the names Q2 8220 and Dow Corning 929 and 939 by the company Dow Corning. The substituted amine groups may be chosen from, for example, C₁-C₄ aminoalkyl groups;
thiol groups, such as the products sold under the names GP 72 A and GP 71 from Genesee;
alkoxylated groups, such as the product sold under the name SILICONE COPOLYMER F-755 by SWS Silicones and ABIL WAX 2428, 2434, and 2440 by the company Degussa;
hydroxylated groups, such as the polyorganosiloxanes comprising a hydroxyalkyl functional group described in French Patent Application No. FR-A-85 16334;
acyloxyalkyl groups, such as the polyorganosiloxanes described in U.S. Pat. No. 4,957,732;
anionic groups of the carboxylic type, such as the products described in European Patent No. EP 186 507 from the company Chisso Corporation, and anionic groups of the alkylcarboxylic type, such as those present in the product X-22-3701 E from the company Shin-Etsu; 2-hydroxyalkyl sulfonate; 2-hydroxyalkyl thiosulfate such as the products sold by the company Degussa under the names ABIL S201 and ABIL S255; and
hydroxyacylamino groups, such as the polyorganosiloxanes described in European Patent Application No. EP 342 834. Non-limiting mention may be made, for example, of the product Q2-8413 from the company Dow Corning.

According to one embodiment, it may be possible to use silicones comprising a polysiloxane portion and a portion comprising a non-silicone organic chain, wherein one of the two portions constitutes the main chain of the polymer and the other is grafted onto the main chain. These polymers are described, for example, in European Patent Application Nos. EP-A-412 704, EP-A-412 707, EP-A-640 105, and EP-A-582 152, in International Patent Publication Nos. WO 95/00578 and WO 93/23009, and in U.S. Pat. Nos. 4,693,935, 4,728,571, and 4,972,037. These polymers may be, for example, anionic or nonionic.

Such polymers may be, for example, copolymers that can be obtained by free-radical polymerization starting with a monomer mixture comprising:
a) 50 to 90% by weight of tert-butyl acrylate;
b) 0 to 40% by weight of acrylic acid;
c) 5 to 40% by weight of silicone macromer of formula: 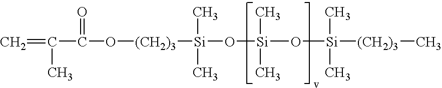
   wherein v is a number ranging from 5 to 700; the weight percentages being calculated relative to the total weight of the monomers.

Other examples of grafted silicone polymers include polydimethylsiloxanes (PDMS) onto which are grafted, via a connecting chain unit of thiopropylene type, mixed polymer units of poly(meth)acrylic acid type and of polyalkyl (meth)acrylate type, and polydimethylsiloxanes (PDMS) onto which are grafted, via a connecting chain unit of thiopropylene type, polymer units of polyisobutyl (meth)acrylate type.

According to the present disclosure, all of the silicones can also be used in the form of emulsions.

The polyorganosiloxanes that may be used in compositions according to the present disclosure may be chosen, for example, from:
non-volatile silicones chosen from the family of polyalkylsiloxanes comprising trimethylsilyl end groups, such as oils having a viscosity ranging from 0.2 to 2.5 m²/s at 25° C., such as the oils of the DC200 series from Dow Corning, for instance, those with a viscosity of 60 000 cSt, of the MIRASIL DM series, for example the oil MIRASIL DM 500 000 sold by the company Rhodia Chimie and the silicone oil AK 300.000 from the company Wacker, polyalkylsiloxanes comprising dimethylsilanol end groups such as dimethiconols, and polyalkylarylsiloxanes, such as the oil MIRASIL DPDM sold by the company Rhodia Chimie; and
polysiloxanes comprising amino groups, such as amodimethicones and trimethylsilylamodimethicones.

According to the present disclosure, the compounds of ceramide type may be chosen from natural and synthetic ceramides, glycoceramides, pseudoceramides, and neoceramides.

The compounds of ceramide type may be chosen, for example, from those described in German Patent Application Nos. DE 4 424 530, DE 4 424 533, DE 4 402 929, and DE 4 420 736, International Patent Publication Nos. WO 95/23807, WO 94/07844, WO 95/16665, WO 94/24097, and WO 94/10131, European Patent Application Nos. EP-A-0 646 572 and EP-A-0 227 994, and French Patent Application No. FR-2 673 179, the contents of which are included herein by way of reference.

The compounds of ceramide type that may be used in compositions according to the present disclosure include, for example:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, for example N-stearoylphytosphingosine,
2-N-palm itoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
   and mixtures of these compounds.

According to the present disclosure, the at least one conditioning agent may be present in an amount ranging from 0.001% to 10% by weight, for example from 0.005% to 5% by weight, such as from 0.01% to 3% by weight, relative to the total weight of the final composition.

The physiologically acceptable medium may be chosen from, for example, water and a mixture of water and at least one solvent chosen from cosmetically and dermatologically acceptable solvents, such as monoalcohols, polyalcohols, and glycol ethers. The water may be present, for example, in an amount ranging from 30% to 98% by weight, for example from 50% to 98% by weight, relative to the total weight of the composition.

For example, the at least one solvent may be chosen from monoalcohols such as ethanol and isopropanol, polyalcohols such as diethylene glycol and glycerol, and polyol ethers such as propylene glycol alkyl ethers.

The compositions according to the present disclosure can also comprise at least one additive chosen from sequestering agents, softeners, foam modifiers, dyes, other nacreous agents, hydrating agents, antidandruff and antiseborrheic agents, other suspension agents, hydroxy acids, thickeners, fatty acid esters, fragrances, preserving agents, sunscreens, proteins, vitamins and provitamins, polymers, and any other additive conventionally used in cosmetics.

The at least one additive may be present in the compositions according to the present disclosure in an amount ranging from 0% to 40% by weight, relative to the total weight of the composition. The precise amount of each additive depends on its nature and is readily determined by a person skilled in the art.

Needless to say, a person skilled in the art will take care to select any optional compound to be added to the compositions according to the present disclosure such that the beneficial properties intrinsically associated with the compositions disclosed herein are not, or are not substantially, adversely affected by the envisaged addition.

The compositions according to the present disclosure may be in the form chosen from gels, milks, creams, more or less thickened lotions, and mousses.

The compositions according to the present disclosure may be used for treating keratin materials such as the hair, the skin, the eyelashes, the eyebrows, the nails, the lips, and the scalp.

The compositions according to the present disclosure may also be used for washing and cleansing keratin materials, such as the hair and the skin.

The compositions according to the present disclosure may be used as products for, for instance, washing, caring for, conditioning, maintaining the hairstyle, and/or shaping keratin materials such as the hair.

For example, the compositions of the present disclosure may be in the form of shampoos, rinse-out or leave-in conditioners, permanent-waving, relaxing, dyeing, and bleaching compositions, in the form of compositions to be applied before or after dyeing, bleaching, permanent-waving and/or relaxing the hair, and in the form of compositions to be applied between the two steps of a permanent-waving or hair-relaxing operation. For example, the compositions may be washing and foaming compositions for the hair and/or the skin.

For example, the compositions according to the present disclosure may be foaming detergent compositions such as shampoos, shower gels, and bubble baths. In this embodiment, the compositions may comprise at least one detergent surfactant.

The at least one detergent surfactant may be chosen from the anionic, amphoteric, nonionic, zwitterionic, and cationic surfactants described above, for example from anionic surfactants and mixtures of anionic surfactants and of amphoteric and/or nonionic surfactants.

The minimum amount of the at least one surfactant is the amount that is just sufficient to give the final composition a satisfactory foaming power and/or detergent power.

Thus, according to the present disclosure, the at least one detergent surfactant may be present in an amount ranging from 3% to 30% by weight, for example from 6% to 25% by weight, such as from 8% to 20% by weight, relative to the total weight of the final composition.

The foaming power of the compositions according to the disclosure, measured by a foam height, may, for example, be greater than 75 mm, such as greater than 100 mm, measured according to the modified Ross-Miles method (NF T 73-404/ISO 696).

The modifications to the method are as follows:

The measurement is performed at a temperature of 22° C. with osmosed water. The concentration of the solution is 2 g/l. The drop height is 1 m. The amount of composition that is dropped is 200 ml. The 200 ml of composition fall into a measuring cylinder 50 mm in diameter and comprising 50 ml of the test composition. The measurement is carried out 5 minutes after stopping the flow of the composition.

The compositions according the present disclosure may also be in the form of rinse-out or leave-in conditioners, permanent-waving, relaxing, dyeing, and bleaching compositions, and in the form of rinse-out compositions, to be applied before or after dyeing, bleaching, permanent-waving or relaxing the hair, or between the two steps of a permanent-waving or hair-relaxing operation.

When the compositions are in the form of a conditioner optionally to be rinsed out, it may also comprise a cationic surfactant in an amount ranging from 0.1% to 1.0% by weight, for example from 0.5% to 5% by weight, relative to the total weight of the composition.

The compositions according to the present disclosure may also be in the form of washing compositions for the skin, for example in the form of bath and shower solutions and gels, and makeup-removing products.

The compositions according to the present disclosure may also be in the form of aqueous and aqueous-alcoholic lotions for skincare and/or haircare.

The cosmetic compositions according to the present disclosure may also be in a form chosen from gels, milks, creams, emulsions, thickened lotions, and mousses, and may be used for the skin, the nails, the eyelashes, the lips, and the hair. The compositions may be liquid.

The compositions according to the present disclosure may be packaged in various forms, for example in vaporizers, pump-dispenser bottles, and aerosol containers so as to allow application of the composition in vaporized form and in mousse form. Such packaging forms are recommended, for example, when it is desired to obtain a spray, a lacquer, or a mousse for treating the hair.

In another aspect, the present disclosure relates to cosmetic processes for treating keratin materials such as the hair, comprising applying to the hair a composition as disclosed herein, optionally rinsing with water after an optional leave-in time.

The present disclosure may be understood more clearly with the aid of the non-limiting examples that follow, which constitute various embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements. In the text hereinbelow, the term “A.M.” is understood to mean Active Material.

### EXAMPLE 1

Two shampoos of compositions A, which is in accordance with the disclosure, and B, which is not in accordance with the disclosure, were prepared:

| Composition | A | | B | |
|---|---|---|---|---|
| Sodium lauryl ether sulfate comprising | 15.5 | g A.M. | 15.5 | g A.M. |
| 2.2 mol of ethylene oxide, at 70% A.M. | | | | |
| Cocoamphodiacetate | 3 | g A.M. | 3 | g A.M. |
| Cyclodextrin, sold under the name | 2 | G | 2 | g |
| Cavamax W7 by the company Wacker | | | | |
| Sodium chloride | 0.3 | G | | — |
| Oxyethylenated (55 EO) propylene | | — | 2.2 | g |
| glycol oleate (Antil 141 liquid from | | | | |
| Goldschmidt) | | | | |
| Preserving agents, fragrance | qs | | qs | |
| pH agent qs | pH 7 | | pH 7 | |
| Demineralized water qs | 100 | G | 100 | g |

Composition A was stable for more than two months at room temperature and at 45° C. It had an attractive nacreous effect and good cosmetic properties.

Composition B was unstable within 48 days at 45° C.

### EXAMPLE 2

The shampoo compositions below were prepared:

| | A | B | C |
|---|---|---|---|
| Demineralized water qs | 100 g | 100 g | 100 g |
| β-Cyclodextrin | 2 g | 2 g | 2 g |
| Sodium lauryl ether sulfate | 22 g | 22 g | 22 g |
| comprising 2.2 mol of | | | |
| ethylene oxide, at 70% A.M. | | | |
| Cocoamidopropylbetaine | 6.4 g | 6.4 g | 6.4 g |
| (47% A.M.) | | | |
| Citric acid qs | pH 7 (±0.2) | pH 7 (±0.2) | pH 7 (±0.2) |
| Preserving agent, fragrance | qs | Qs | qs |
| Magnesium gluconate | 3.5 g | | |
| Calcium gluconate | | 3.5 g | |
| Potassium dihydrogen | | | 2.9 g |
| phosphate | | | |

Compositions A, B and C were stable and had an attractive nacreous effect. They also had good cosmetic properties.

1. A cosmetic composition comprising, in an aqueous medium,
at least one anionic surfactant in an amount ranging from 1% to 35% by weight, relative to the total weight of the composition,
at least one compound chosen from cyclodextrins and derivatives thereof in an amount ranging from 1% to 15% by weight, relative to the total weight of the composition, and
at least one salt chosen from ammonium (NH₄⁺) salts and salts of monovalent or divalent metals and of a mineral acid or of an organic acid comprising from 1 to 7 carbon atoms in an amount ranging from 0.1% to 10% by weight, relative to the total weight of the composition.

2. The composition according to claim 1, wherein the at least one compound is chosen from α-cyclodextrin, β-cyclodextrin, and γ-cyclodextrin, and derivatives thereof.

3. The composition according to claim 2, wherein the at least one compound is chosen from β-cyclodextrin and γ-cyclodextrin.

4. The composition according to claim 1, wherein the at least one compound is present in an amount ranging from 1% to 10% by weight, relative to the total weight of the final composition.

5. The composition according to claim 4, wherein the at least one compound is present in an amount ranging from 1.5% to 5% by weight, relative to the total weight of the final composition.

6. The composition according to claim 1, wherein the at least one anionic surfactant and the at least one compound are present in amounts effective to allow the formation of an insoluble complex in the composition, and/or to make the composition nacreous.

7. The composition according to claim 1, wherein the salts of monovalent or divalent metals are chosen from alkali metal salts, alkaline-earth metal salts, and transition metal salts.

8. The composition according to claim 7, wherein the salts of monovalent or divalent metals are chosen from lithium, sodium, potassium, magnesium, calcium, strontium, barium, manganese, cobalt, and zinc salts, and mixtures thereof.

9. The composition according to claim 8, wherein the metal salts are chosen from sodium, potassium, magnesium, calcium, manganese, and zinc salts, and mixtures thereof.

10. The composition according to claim 1, wherein the mineral or organic acids are chosen from carbonates, bicarbonates, sulfates, glycerophosphates, borates, chlorides, nitrates, acetates, hydroxy acids, fruit acids, and amino acids.

11. The composition according to claim 1, wherein the at least one salt is chosen from sodium chloride, potassium chloride, calcium chloride, magnesium sulfate, magnesium gluconate, calcium gluconate, and sodium citrate.

12. The composition according to claim 11, wherein the at least one salt is sodium chloride.

13. The composition according to claim 1, wherein the at least one salt is present in an amount ranging from 0.5% to 5% by weight, relative to the total weight of the composition.

14. The composition according to claim 1, wherein the at least one anionic surfactant is present in an amount ranging from 4% to 35% by weight, relative to the total weight of the composition.

15. The composition according to claim 14, wherein the at least one anionic surfactant is present in an amount ranging from 8% to 30% by weight, relative to the total weight of the composition.

16. The composition according to claim 1, further comprising at least one conditioning agent.

17. The composition according to claim 16, wherein the at least one conditioning agent is chosen from poly-α-olefins, fluoro oils, fluoro waxes, fluoro gums, carboxylic acid esters, silicones, cationic polymers, mineral, plant and animal oils, ceramides, pseudoceramides, and mixtures thereof.

18. The composition according to claim 16, wherein the at least one conditioning agent is present in an amount ranging from 0.001% to 10% by weight, relative to the total weight of the composition.

19. The composition according to claim 18, wherein the at least one conditioning agent is present in an amount ranging from 0.005% to 5% by weight, relative to the total weight of the composition.

20. The composition according to claim 19, wherein the at least one conditioning agent is present in an amount ranging from 0.01% to 3% by weight, relative to the total weight of the composition.

21. The composition according to claim 1, wherein the cosmetic composition is in a form chosen from a gel, a milk, a cream, a lotion, and a mousse.

22. The composition according to claim 1, wherein the cosmetic composition is a foaming detergent composition.

23. The composition according to claim 22, wherein the foaming detergent composition is chosen from shampoos, shower gels, and bubble baths.

24. The composition according to claim 1, wherein the composition is chosen from rinse-out and leave-in conditioning compositions; permanent-waving, relaxing, dyeing and bleaching compositions; compositions to be applied before or after dyeing, bleaching, permanent-waving and relaxing the hair; and compositions to be applied between the two steps of a permanent-waving or hair-relaxing operation.

25. A method for treating keratin materials, comprising:
applying to the keratin materials a cosmetic composition comprising, in an aqueous medium,
at least one anionic surfactant in an amount ranging from 1% to 35% by weight, relative to the total weight of the composition,
at least one compound chosen from cyclodextrins and derivatives thereof in an amount ranging from 1% to 15% by weight, relative to the total weight of the composition, and
at least one salt chosen from ammonium (NH₄+) salts and salts of monovalent or divalent metals and of a mineral acid or of an organic acid comprising from 1 to 7 carbon atoms in an amount ranging from 0.1% to 10% by weight, relative to the total weight of the composition; and
optionally rinsing the keratin materials with water.

26. A method for making a cosmetic composition for treating keratin materials, comprising including at least one salt chosen from ammonium (NH₄⁺) salts and salts of a monovalent or divalent metal cation in a composition comprising at least one anionic surfactant and at least one compound chosen from cyclodextrins and derivatives thereof, wherein the at least one salt acts as an agent for suspending a complex formed from the at least one compound and the at least one surfactant.
